(19) United States
(12) Patent Application Publication
(10) Pub. No.: US 2016/0346315 A1
(43) Pub. Date: Dec. 1, 2016

(54) Methods for promoting wound healing

(71) Applicant: UNIVERSITY OF SOUTHERN CALIFORNIA, Los Angeles, CA (US)

(72) Inventors: Kathleen E. Rodgers, Long Beach, CA (US); Gere S. Dizerega, San Luis Obispo, CA (US)

(21) Appl. No.: 15/117,501

(22) PCT Filed: Feb. 12, 2015
(86) PCT No.: PCT/US2015/015555

§ 371 Date: Aug. 9, 2016

Related U.S. Application Data
(60) Provisional application No. 61/938,847, filed on Feb. 12, 2014.

(51) Int. Cl. A61K 31/717; A61K 31/235; A61L 15/44; A61K 9/06; A61K 9/00

(52) U.S. Cl. CPC A61K 31/717; A61K 9/06; A61K 9/0014; A61L 15/44; A61K 31/235; A61L 2300/232

(57) ABSTRACT

The present invention provides methods for promoting wound healing or treating dermal radiation injury by the use of a gel formulation comprising about 0.5% to about 4% hydroxyethyl cellulose (HEC) on a weight (mg)/volume (ml) basis, or on a weight/weight (mg) basis.

### CROSS REFERENCE

This application claims priority to U.S. Provisional Patent Application Ser. No. 61/93,884 filed Feb. 12, 2014, incorporated by reference herein in its entirety.

### BACKGROUND

Epithelial damage following injury can result in lesions from direct exposure or indirectly by damage to epithelial progenitor cells that would otherwise contribute to healing and inflammation. Methods for limiting development of skin wounds are needed in the art.

### SUMMARY OF THE INVENTION

In one aspect, the invention provides methods for promoting wound healing in a subject, comprising contacting a skin wound suffered by a subject with a gel formulation comprising about 0.5% to about 4% hydroxyethyl cellulose (HEC) on a weight (mg)/volume (ml) basis, or on a weight/weight (mg) basis, for a time sufficient to promote healing of the dermal wound, wherein the gel formulation does not include any other active ingredient for treating wounds. In one embodiment, promoting healing of the dermal wound comprises one or more of increasing the rate or completeness of wound healing compared to control, reducing scarring, decreasing the amount or severity of skin lesions, delaying the onset of lesions in response to injury, improved epithelial integrity, and/or reducing inflammation and/o depth of collagen necrosis at the wound site. In another embodiment, the dermal wound is caused by exposure to toxic agents. In a further embodiment, the dermal wound is an incision caused by an event selected from the group consisting of disease, disability, sharp objects, lacerations, burns, abrasions, avulsions, penetration wounds, radiation injury, and gunshot wounds. In further embodiments, the dermal wound comprises diabetic ulcers or bedsores. In another embodiment, the wound is caused by radiation injury, including, but not limited to, radiation injury is caused ultraviolet light, X-rays, microwaves, radio-frequency waves, electromagnetic radiation, therapeutic or accidental X-ray, gamma ray, or beta particle exposure, clinical radiation therapy, medical diagnostics using radioactive tracers, exposure to naturally occurring ionizing radiation sources such as uranium and radon, wartime exposure (nuclear weapons), and accidental exposures including occupational exposure at nuclear power facilities, and medical and research institutions.

In another aspect, the invention provides methods for treating dermal radiation injury, comprising contacting a dermal radiation injury site in a subject with a gel formulation comprising about 0.5% to about 4% hydroxyethyl cellulose (HEC) on a weight (mg)/volume (ml) basis, or on a weight/weight (mg) basis, for a time sufficient to treat the dermal radiation injury. In one embodiment, the dermal radiation injury is caused by radiation selected from the group consisting of ultraviolet light X-rays, microwaves, radio-frequency waves, electromagnetic radiation, therapeutic or accidental X-ray, gamma ray, or beta particle exposure, clinical radiation therapy, medical diagnostics using radioactive tracers, exposure to naturally occurring ionizing radiation sources such as uranium and radon, wartime exposure (nuclear weapons), and accidental exposures including occupational exposure at nuclear power facilities, and medical and research institutions.

In various embodiments of any aspect of the invention the formulation comprises about 1% to about 3% hydroxyethyl cellulose (HEC) on a weight (mg)/volume (ml) basis, or on a weight/weight (mg) basis, or about 2% hydroxyethyl cellulose (HEC) on a weight (mg)/volume (ml) basis, or on a weight/weight (mg) basis.

In a third aspect, the invention provides formulations comprising

(a) about 0.5% to about 4% hydroxyethyl cellulose (HEC) on a weight (mg)/volume (ml) basis, or on a weight/weight (mg) basis, and

(b) about 0.2% to about 0.01% paraben;

wherein the formulation dos not include any other therapeutic.

In one embodiment, the formulation comprises about 1% to about 3% hydroxyethyl cellulose (HEC) on a weight (mg)/volume (ml) basis, or on a weight/weight (mg) basis. In another embodiment, the formulation comprises about 2% hydroxyethyl cellulose (HEC) on a weight (mg)/volume (ml) basis, or on a weight/weight (mg) basis. In a further embodiment, the paraben is selected from the group consisting of methyl paraben, butyl paraben, ethyl paraben, heptyl paraben, and propyl paraben. In another embodiment, the formulation comprises about 0.11% methyl paraben on a weight (mg)/volume (ml) basis, or on a weight/weight (mg) basis. In a further embodiment, the formulation comprises about 0.022% propyl paraben on a. weight (mg)/volume (ml) basis, or on a weight/weight (mg) basis. In various further embodiments, the formulation is present in a syringe of catheter, and/or the formulation is provided on a wound dressing.

### DESCRIPTION OF THE FIGURES

FIG. 1. Necropsy diagram of guinea pig skin. The center point of the diagonal cross axes (blue) will be used to determine the internal 2 cm×2 cm area. For the internal area quadrants (designated LESION) B and D will be fixed in formalin for histological preparation and quadrants A and C will be placed in RNALater® for gene expression analysis. The remaining area of radiation exposure (outside of the internal 2 cm×2 cm area, designated EDGE) will be cut into 4 pieces, two placed in formalin for histological preparation (areas 3 and 4) and two in RNALater® for gene expression analysis (areas 1 and 2).

FIG. 2. Graph of HEC treatment effect on radiation injury generated by 36 Gy irradiation, as measure by skin lesions using the Kumar Score.

FIG. 3. Graph of HEC treatment effect on radiation injury generated by 41 Gy irradiation, as measure by skin lesions using the Kumar Score.

FIG. 4(A-E). Tables showing HEC treatment effect on various measures of dermal injury caused by 32 Gy irradiation treatment on (a) epithelial integrity; (b) upper dermis inflammation; (c) inflammation above the adipose tissue layer; (d) collagen necrosis, and (e) blood vessel granulation.

FIG. 5(A-E). Tables showing HEC treatment effect on various measures of dermal injury caused by 36 Gy irradiation treatment on (a) epithelial integrity; (b) upper dermis inflammation; (c) inflammation above the adipose tissue layer; (d) collagen necrosis, and (e) blood vessel granulation.

FIG. 6(A-E). Tables showing HEC treatment effect on various measures of dermal injury caused by 41 Gy irradiation treatment on (a) epithelial integrity; (b) upper dermis inflammation; (c) inflammation above the adipose tissue layer; (d) collagen necrosis, and (e) blood vessel granulation.

### DETAILED DESCRIPTION OF THE INVENTION

As used herein, the singular forms “a,” “an” and “the” include plural referents unless the context clearly dictates otherwise. “And” as used herein is interchangeably used with “or” unless expressly stated otherwise.

All embodiments within and between different aspects of the invention can be combined unless the context clearly dictates otherwise.

As used herein, the term “about” means +/−5% of the relevant measurement or unit.

In a first aspect, the invention provides methods for promoting wound healing in a subject, comprising contacting a skin wound suffered by the subject with a gel formulation comprising about 0.5% to about 4% hydroxyethyl cellulose (HEC) on a weight (mg)/volume (ml) basis, or on a weight/weight (mg) basis, for a time sufficient to treat the dermal wound, wherein the gel formulation does not include any other active ingredient for treating wounds. in one embodiment, the formulation comprises about 1% to about 3% hydroxyethyl cellulose (HEC) on a weight (mg)/volume (ml) basis, or on a weight/weigh((mg) basis. In another embodiment, the formulation comprises about 2% hydroxyethyl cellulose (HEC) on a weight (mg)/volume (ml) basis, or on a weight/weight (mg) basis.

The inventors have unexpectedly discovered that gel formulations as recited above can be used to promote wound healing in the absence of other therapeutics, and that they work significantly better than the current standard of case (Aloe Vera, water, soap, petrolatum). The methods of the invention can be used to promote healing of any wound, where “promoting healing” comprises providing any clinical benefit, including but not limited to increased rate or completeness of wound healing compared to control, reduced scarring, decreased amount or severity of skin lesions, delay the onset of lesions in response to injury, improved epithelial integrity, reduced inflammation and/or depth of collagen necrosis at the wound site, etc.

The subject may be any suitable subject, including but not limited to humans, dogs, cats, cattle, horses, goats, sheep, pigs, other mammals, chickens, etc.

The formulation for use in wound healing may have any other inactive components as deemed appropriate for a given use, including but not limited preservatives. In one exemplary embodiment, a paraben is added as to the HEC formulation as a preservative. Such parabens include, but are not limited to parahydroxybenzoates, esters of parahydroxybenzoic acid (also known as 4-hydroxybenzoic acid), or salts thereof, such as ethyl paraben, butyl paraben, ethyl paraben, heptyl paraben, and propyl paraben. Such parabens can be added at any suitable amount as deemed appropriate by an attending physician depending on all circumstances, in various embodiments, the HEC formulation may include about 0.2% to about 0.01% paraben; about 0.15% to about 0.02% paraben, or about 0.12% to about 0.022% paraben (all w/w w/v). In non-limiting examples, the HEC formulation may comprise about 0.11% methyl paraben or about 0.022% propyl paraben.

The formulation is administered topically to the wound (and may also be applied to surrounding, non-wounded dermal tissue) in an appropriate amount/volume as necessitated by the size and severity of the wound, as well as specifics of the wound to be treated. in various non-limiting embodiments, the formulation can be administered via syringe, catheter, swab, tube, dropper, or gloved hand; followed by spreading the formulation over the wound area as appropriate, and applying a bandage and tape as appropriate. As will be understood by those of skill in the art, the formulation may be applied for a limited period of time, removed, and the formulation reapplied, with this process being repeated as frequently as deemed appropriate,

The wound may be one caused by any type of jury. In one embodiment, the dermal wound is caused by exposure to a toxic agent. Examples of such toxic agents include, but are not limited to allergens (food, dyes, medicine, insect bites or stings, metals, etc.), skin contact with an irritant (chemical agent, mechanical (clothing, etc.), thermal, radiative etc.), chemical exposure (including but not limited to cosmetic, detergent, solvent, etc.), contact dermatitis, vesicants (including but not limited to mustard gas and Lewisite); exposure to poison ivy, exposure to poison oak; bacteria, and viruses (such as Herpes Simplex). in another embodiment, the dermal wound is an incision caused by an event selected from the group consisting of disease, disability, sharp objects, lacerations, burns, abrasions, avulsions, penetration wounds, radiation injury, and gunshot wounds. in a further embodiment, the dermal wound comprises a diabetic ulcer, in another embodiment, the dermal wound comprises bedsores.

In another embodiment. the wound is caused by radiation injury. Such radiation injury may be caused by radiation including but not limited to ultraviolet light, X-rays, microwaves, radio-frequency waves, electromagnetic radiation, therapeutic or accidental X-ray, gamma ray, or beta particle exposure, clinical radiation therapy, medical diagnostics using radioactive tracers, exposure to naturally occurring ionizing radiation sources such as uranium and radon, wartime exposure (nuclear weapons, dirty bomb, etc.), and accidental exposures including occupational exposure at nuclear power facilities, nuclear power plant fallout, industrial accident, and medical and research institutions In one embodiment, the subject has been exposed to total body ionizing irradiation of between 0.2 gray Gy to 12 Gy or greater; in further embodiments the subject has been exposed to total body ionizing irradiation of between 1 gray Gy to 12 Gy or greater; 2 gray y to 12 Gy or greater; 0.2 Gy to 10 Gy or greater; 1 Gy to 10 Gy or greater; 2 gray (G)y to 10 Gy or greater; 2.5 Gy to 10 Gy or greater; 3 Gy to 10 Gy or greater; 3.5 Gy to 10 Gy or greater; 4 Gy to 10 Gy or greater; 4.5 Gy to 10 Gy or greater.; 5 Gy to 10 Gy or greater; 5.5 Gy to 10 Gy or greater; 6 Gy to 10 Gy or greater; 6.5 Gy to 10 Gy or greater; 7 Gy to 10 Gy or greater; 7.5 Gy to 10 Gy or greater; 8 Gy to 10 Gy or greater; 8.5 Gy to 10 Gy or greater; 9 Gy to 10 Gy or greater; greater than 10 Gy; or greater than 12 Gy. In another embodiment, the subject has suffered cumulative exposure to total body ionizing irradiation of at least 20 cGy. In various further embodiments, the subject has suffered cumulative exposure to total body ionizing irradiation of at least 25 cGy, 30 cGy, 35 cGy, 40 cGy, 45 cGy, 50 cGy, 55 cGy, 60 cGy, 65 cGy, 70 cGy, 75 eGy, 80 cGy, 85 cGy, 90 eGy, 95 cGy, 100 eGy, or greater.

The burn may be of any severity, preferably a partial thickness burn (i.e.: second-degree burn) to any dermal site, including but not limited to trunk, back, head, arm, or leg. The burn may be of any size, preferably at least 3 cm² in area, and more preferably at least 4, 5, 6, 8, 9, or 10 cm² in area. In a further embodiment, the subject has suffered burns (such as second degree burns) over at least 10%, 20%, 30%, 40%, 50%, 60% 79%, or more of their total body surface area.

In another aspect, the invention provides methods for treating dermal radiation injury, comprising contacting a dermal radiation injury site with a gel formulation comprising about 0.5% to about 4% hydroxyethyl cellulose (HEC) on a weight (mg)/volume (ml) basis, or on a weight/weight (mg) basis, for a time sufficient to treat the dermal radiation injury.

The formulation for use in treating dermal radiation injury may have any other active or inactive components as deemed appropriate for a given use. In one embodiment, the gel formulation does not include any other active ingredient for treating dermal radiation injury. In another embodiment, the gel formulation includes one or more other active ingredient, such as antimicrobials, preservatives or anti-inflammatory agents, for treating dermal radiation injury. All embodiments disclosed above for the first aspect of the invention can also be used in this second aspect of the invention.

In a third aspect, the invention provides formulations comprising:

(a) about 0.5% to about 4% hydroxyethyl cellulose (HEC) on a weight (mg)/volume (ml) basis, or on a weight/weight (mg) basis, and

(b) about 0.2% to about 0.01% paraben;

wherein the formulation dos not include any other therapeutic.

In various embodiments, the formulation comprises about 1% to about 3% hydroxyethyl cellulose (HEC) on a weight (mg)/volume (ml) basis, or on a weight/weight (mg) basis, or about 2% hydroxyethyl cellulose (HEC) on a weight (mg)/volume (ml) basis, or on a weight/weight (mg) basis. In various other embodiments, the formulation comprises about 0.2% to about 0.01% paraben; about 0.15% to about 0.02% paraben, or about 0.12% to about 0.022% paraben (all w/w or w/v). In non-limiting examples, the formulation may comprise about 0.11% methyl paraben or about 0.022% propyl paraben.

In another embodiment, the formulation is provided in a syringe or catheter. In a further embodiment, the formulation is provided on a wound dressing (i.e.: bandage, medical tape, semipermeable films, foams, hydrocolloids, plastic or synthetic dressing, wound cover, and calcium alginate swabs).

### EXAMPLES

Epithelial injury following low-penetrating radiation can result in lesions from direct exposure or indirectly by damage to epithelial progenitor cells that would otherwise contribute to healing and inflammation, A guinea pig model was developed utilizing the XRAD320ix Biological Irradiator with the instrument collimator removed and an aperture defined by lead shielding used as an expeditious first pass surrogate for beta radiation. The low-penetrating radiation was used for acute high dose exposure of the skin as might occur during radiation contamination of the skin. In this study, the effect of treatment of a radiation injury with Aloe Vera and bandaging (standard of care) or 2% hydroxyethyl cellulose in 0.05 M phosphate buffer and parabens and bandaging was determined

The purpose of this protocol was to perform studies to characterize the effect of bandaging and treatment with hydrogels (Aloe Vera or vehicle) on the cutaneous response and in-life injury phase of guinea pigs after exposure to low-penetrating radiation. After exposure to a blast containing radioactive material, dermal injuries can arise in which healing is impaired due to radiation injury of dermal and epidermal progenitor cells and chronic inflammation.

The guinea pig skin model was developed using 50 kVp x--rays exposure via the XRAD320ix Biological Irradiator. The XRAD320ix was configured without a filter and the instrument collimator removed. This exposure mode acts as a high throughput surrogate for low-penetrating radiation (similar to beta energy). The exposure was on the left side of the guinea pig starting at the forward leg and moving caudally for 4 cm. In this study, the area exposed to the attenuated x-ray was defined by lead shielding which allows a 4 cm by 4 cm field of exposure.

We utilized the guinea pig, whose skin architecture and hair growth pattern closely resembles that of humans. The radiation source, 50 kVp x-rays with no filtration and no instrument collimator, was chosen as a high throughput surrogate for low-penetrating radiation. The 50 kVp x-rays with no filtration and no collimator have a HVL of 0.12 mm of aluminum. The features of this system include a high dose rate (5.93 Gy/minute), good homogeneity (1.2% variance over a central 3.0 cm×3.0 cm area), consistent dose rate over time (0.9% variance), good dose homogeneity over time (1.5% variance), a shallow deposition of the ionization dose with depth (measured in tissue equivalent plastic at a depth of 2.1 mm to be 50% that of the surface dose), and dose linearity (<0.1% with exposures up to 30 Gy).

Male guinea pigs (Hartley strain) between 450-550 grams in weight were obtained from Charles Rivers, and were subjected to exposure of the upper left body starting at the forward leg, approximately 0.3 cm from the spine extending 4 cm in each direction. Fur was removed from the animal's left side by light shaving followed by depilation with Nair for three minutes (removal with warm water) to allow hair removal without nicking the skin. The guinea pig was lightly anesthetized/immobilized with Ketamine/Xylazine (40/4 mg/kg), positioned, and irradiated to the prescribed absorbed dose. The low penetrating x-ray (50 kVp at 30 mAmps, 5.93 Gy/minute via, 50 cm SSD, on the XRAD320ix X-Ray without filter and collimator) was localized in a 4 cm by 4 cm area using a 4 mm thick lead shield. Animals were housed one to a cage, fed standard laboratory chow, and provided water ad libitum.

The purpose of the study was to characterize changes that occur to the cutaneous response to low-penetrating radiation injury (erythema, desquamation, induration, etc) when the site was exposed to hydrogels (Aloe Vera as the care normally given to patients in the clinical setting and 2% HEC) and bandaging.

All studies were performed using guinea pigs with only hydrogels and bandaging as medical interventions. The cutaneous response was characterized in animals at the following radiation doses and exposure to hydrogels and bandaging was initiated at erythema or loss of dermal integrity. Clinical endpoints of the cutaneous response to radiation (erythema, desquamation, and induration) were used to define the response profile. The scoring system is found on Table 1 for the RTOG system and Table 2 for the Kumar system.

TABLE 1
RTOG ® Acute Radiation Morbidity Scoring Criteria for Skin

| Score | Skin Changes |
|---|---|
| 0 | No change over baseline |
| 1 | Follicular, faint or dull erythema/epilation/dry desquamation/ |
| | decreased sweating |
| 2 | Moderate to brisk erythema, tender or bright erythema, patchy |
| | moist desquamation/moderate edema |
| 3 | Confluent, moist desquamation not confined to the skin folds. |
| | Pitting edema (sever swelling). |
| 4 | Life-threatening consequences; skin necrosis or ulceration of |
| | full thickness dermis; hemorrhage; spontaneous bleeding from |
| | involved site; skin graft indicated |
| 5 | Death |

Table 1. Assessment Criteria: RTOG Scale utilized to score o finical endpoints of the cutaneous response to radiation.

TABLE 2
Kumar Scale

| Score | Skin changes |
|---|---|
| 1.0 | No Effect |
| 1.5 | Minimal erythema, mild dry skin |
| 2.0 | Moderate erythema, dry skin |
| 2.5 | Marked erythema, dry desquamation |
| 3.0 | Dry desquamation, minimal dry crusting |
| 3.5 | Dry desquamation, dry crusting, superficial minimal scabbing |
| 4.0 | Patchy, moist desquamation, moderate scabbing |
| 4.5 | Confluent moist desquamation, ulcers, large deep scabs |
| 5.0 | Open wound, full thickness skin loss |
| 5.5 | Necrosis |

Table 2. Assessment Criteria: Kumar Scale utilized to score clinical endpoints of the cutaneous response to radiation.

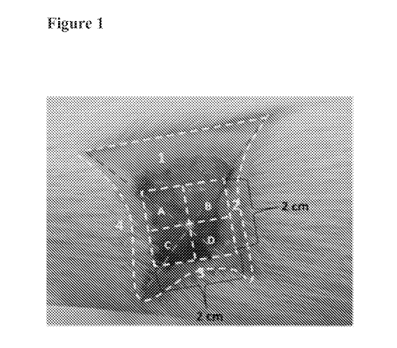

The area irradiated was on the left side of the guinea pig starting from the front leg, approximately 0.3 cm from the spine. Within the central 2 cm by 2 cm of this area, there were 4 quadrants assessed (FIG. 1). The exposure area was traced on the day of irradiation and the corners were tattooed. Upon necropsy, the guinea pig tissue was harvested, divided as shown in FIG. 1, and used for histological analysis.

Treatment with Aloe Vera or 2% hydroxyethyl cellulose (HEC) with 0.11% methyl paraben or 0.022% propyl paraben was initiated at a predetermined trigger corresponding to erythema (Kumar score of the group of 1.5 or greater) or loss of dermal integrity (LDI Kumar score of the group of 3.5 or greater). 1.2 ml of treatment was administered over the 16 cm² area of radiation exposure using a 3 mL syringe daily until necropsy. Investigational agent and bandaging were performed as follows:

1) The gel was applied over the wound area via a catheter and then the catheter was used to spread the gel to a thin layer over the entire wound area.

2) Once the gel was applied, the wound was covered with a double layer of Tegaderm® and overwrapped with Elastikon® tape. The double layer of Tegaderm® was layered so that the adhesive sides of the Tegaderm® face each other.

3) Before each treatment, the bandage was removed from the wound area and moist gauze was used to gently remove any remaining gel from the previous application. The gel was then re-applied as described above.

All of the animals were observed for clinical signs of abnormality immediately after exposure to radiation and daily until the end of necropsy. The irradiated area was assessed daily for changes in clinical endpoints and scored using the Kumar Scale (Table 1) (Kumar et al, 2008). The wound area was visually assessed as four individual quadrants. The highest quadrant score became the overall score for the wound for that day. At necropsy (day 30), the inner 2 cm by 2 cm of the irradiated area (4 cm by 4 cm) was cut into 4 (four) quadrants. The inner 2 cm by 2 cm area was measured based upon the center point of the cross axes of the initial corner marks. For the internal area quadrants (designated LESION) B and D were fixed in formalin for histological preparation and quadrants A and C were placed in RNALater® for gene expression analysis. The remaining area of radiation exposure (outside of the internal 2 cm×2 cm area, designated EDGE) was cut into 4 pieces, two placed in formalin for histological preparation (areas 3 and 4) and two in RNALater® for gene expression analysis (areas 1 and 2).

Hematoxylin Eosin Stains: Formalin-fixed, paraffin-embedded tissue sections were mounted on slides, deparaffinized in an organic solvent, and cleared into water with graded concentrations of alcohol. Slides were immersed and over-stained with Mayer's hematoxylin and excess staining was removed by immersion into an alcoholic/acidic solution. The de-staining was halted by immersion in an alkaline solution. The slides were then counterstained with eosin dye followed by immersion in graded alcohols and clearing of aqueous reagent with organic solvents followed by placement of a coverslip with epoxy resin.

Hematoxylin and eosin stained slides were evaluated for qualitative changes in several measures including epithelial, dermal and inflammatory changes. The changes were assigned scores from 0-3 (0=no change, 1=mild, 2=moderate, and 3=severe), unless otherwise noted. The variations from this system included a grade of 4 for epithelial changes if the epithelium is absent. The other variation from this system was with regards to the depth of collagen necrosis. The depth of necrosis will be measured as none (0), superficial (1), to the bottom of the hair follicle (2), mid in the dermis (3) or to the adipose layer (4).

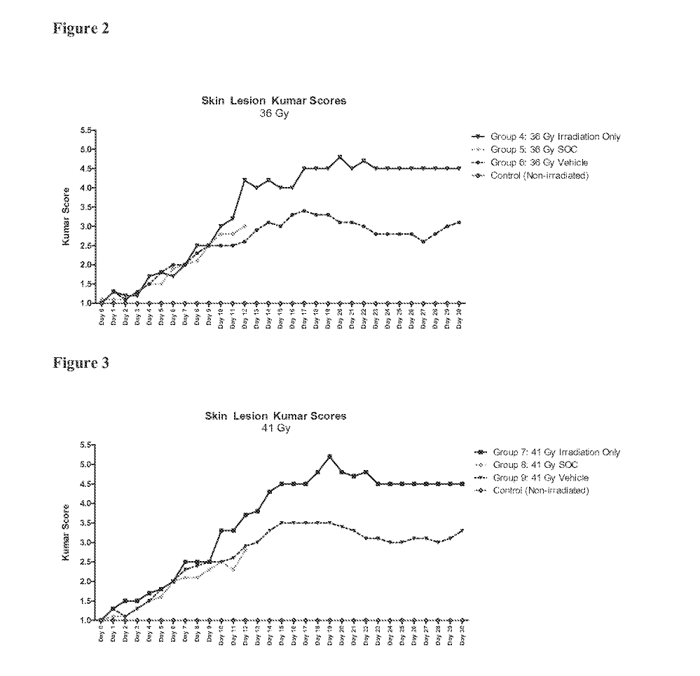

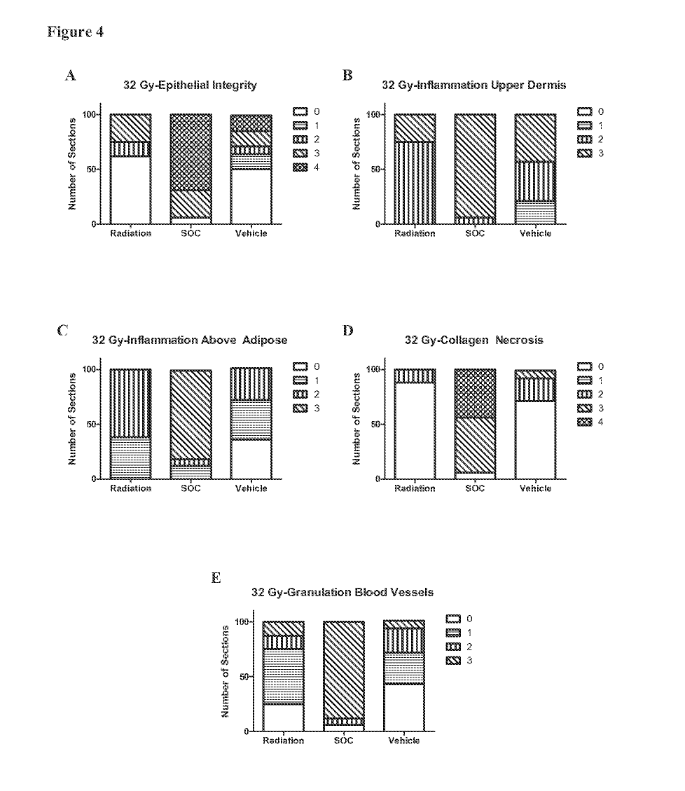

Treatment of radiation burns generated by 36 or 41 Gy irradiation with 2% HEC gel with parabens (vehicle) significantly decreased the skin lesions as measured by Kumar Score (FIGS. 2-3). Further, treatment with vehicle was better than the current standard of care for radiation burns, Aloe Vera in the reduction of skin lesions, and the radiation only groups.

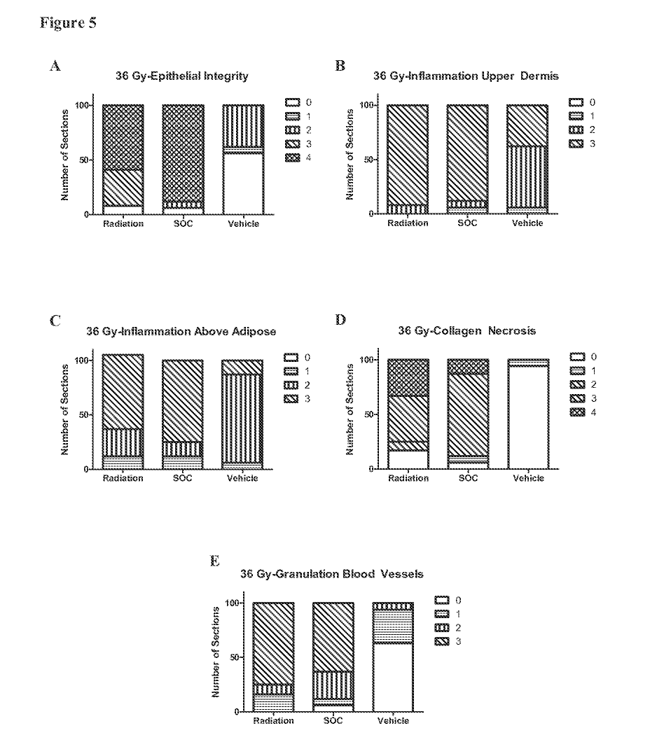

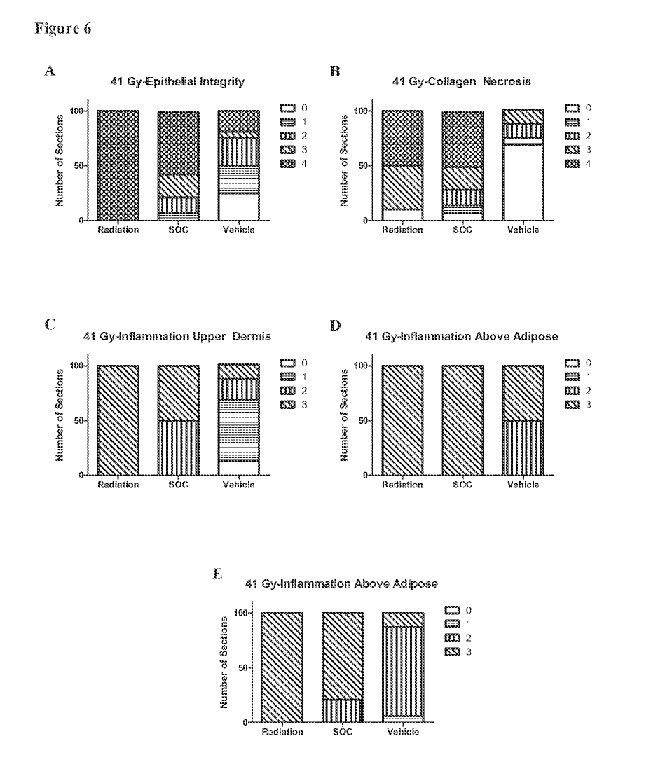

The benefit to utilization of the vehicle in comparison to standard of care was inure obvious after histological examination of the tissues (FIGS. 4-6). The epithelial integrity was significantly improved in the vehicle treated group compared with the standard of care (Aloe Vera). Similarly, changes in the formation of granulation tissue at the irradiated site, inflammation at several levels in the dermis, and the depth of collagen necrosis was significantly reduced in the sites treated with vehicle compared with sites treated with. SOC and radiation only. After day 12, the Aloe Vera (standard of care) was difficult to remove and the wound could not be visualized. Thus, SOC data in the graph is only provided through day 12. However, when the tissues were evaluated histologically it became clear that the tissues treated with Aloe Vera would have showed much more severe scores if the wound could have been observed.

### References

Kumar, S., Kolozsvary, A., Kohl, R., Lu, M., Brow/ S., & Kim, J. H. (2008). Radiation-induced skin injury in the animal model of scleroderma: implications for post-radiotherapy fibrosis. Radiation Oncology, 3:40-47.

Ouhtit, A., Muller, H. K., Davis, D. W., Ullrich, S. E., McConkey, D., & Ananthaswamy, H. N. (2000). Temporal events in skin injury and the early adaptive responses in ultraviolet-irradiated mouse skin. Am J Pathol. 156(1):201-207.

Withers H R, Flow B L, Huchton J I, Hussey D H, Jardin J H, Mason K A, Raulston G L, Smathers J B. (1977). Effect of dose fractionation on early and late skin responses to γ-rays and neutrons. Intl, Journal. Radiation Oncology Biol. Phys, 3:227-233.

1. A method for promoting wound healing in a subject, comprising contacting a skin wound suffered by a subject with a gel formulation comprising about 0.5% to about 4% hydroxyethyl cellulose (HEC) on a weight (mg)/volume (ml) basis, or on a weight/weight (mg) basis, for a time sufficient to promote healing of the dermal wound, wherein the gel formulation does not include any other active ingredient for treating wounds.

2. The method of claim 1, wherein promoting healing of the dermal wound comprises one or more of increasing the rate or completeness of wound healing compared to control, reducing scarring, decreasing the amount or severity of skin lesions, delaying the onset of lesions in response to injury, improved epithelial integrity, and/or reducing inflammation and/or depth of collagen necrosis at the wound site.

3. The method of claim 1, wherein the dermal wound is caused by exposure to toxic agents.

4. The method of claim 1, wherein the dermal wound is an incision caused by an event selected from the group consisting of disease, disability, sharp objects, lacerations, burns, abrasions, avulsions, penetration wounds, radiation injury, and gunshot wounds.

5. The method of claim 1, wherein the dermal wound comprises diabetic ulcers.

6. The method of claim 1, wherein the dermal wound comprises bedsores.

7. The method of claim 1, wherein the wound is caused by radiation injury.

8. The method of claim 7, wherein the radiation injury is caused by radiation selected from the group consisting of ultraviolet light, X-rays, microwaves, radio-frequency waves, electromagnetic radiation, therapeutic or accidental X-ray, gamma ray, or beta particle exposure, clinical radiation therapy, medical diagnostics using radioactive tracers, exposure to naturally occurring ionizing radiation sources, wartime exposure, and accidental exposures.

9. A method for treating dermal radiation injury, comprising contacting a dermal radiation injury site in a subject with a gel formulation comprising about 0.5% to about 4% hydroxyethyl cellulose (HEC) on a weight (mg)/volume (ml) basis, or on a weight/weight (mg) basis, for a time sufficient to treat the dermal radiation injury.

10. The method of claim 9 wherein the dermal radiation injury is caused by radiation selected from the group consisting of ultraviolet light, X-rays, microwaves, radio-frequency waves, electromagnetic radiation, therapeutic or accidental X-ray, gamma ray, or beta particle exposure, clinical radiation therapy, medical diagnostics using radioactive tracers, exposure to naturally occurring ionizing radiation sources, wartime exposure, and accidental exposures.

11. The method of claim 1, wherein the formulation comprises about 1% to about 3% hydroxyethyl cellulose (HEC) on a weight (mg)/volume (ml) basis, or on a weight/weight (mg) basis.

12. The method of claim 1, wherein the formulation comprises about 2% hydroxyethyl cellulose (HEC) on a weight (mg)/volume (ml) basis, or on a weight/weight (mg) basis.

13. The method of claim 1, wherein the formulation comprises paraben.

14. The method of claim 13, wherein the paraben is selected from the group consisting of methyl paraben, butyl paraben, ethyl paraben, heptyl paraben, and propyl paraben.

15. The method of claim 1, wherein the formulation comprises about 0.2% paraben to about 0.01% paraben on a weight (mg)/volume (ml) basis, or on a weight/weight (mg) basis.

16. The method of claim 1 wherein the formulation comprises about 0.11% methyl paraben on a weight (mg)/volume (ml) basis, or on a weight/weight (mg) basis.

17. The method of claim 1 wherein the formulation comprises about 0.022% propyl paraben on a weight (mg)/volume (ml) basis, or on a weight/weight (mg) basis.

18. A formulation comprising
(a) about 0.5% to about 4% hydroxyethyl cellulose (HEC) on a weight (mg)/volume (ml) basis, or on a weight/weight (mg) basis, and
(b) about 0.2% to about 0.01% paraben;
wherein the formulation dos not include any other therapeutic.

19. The formulation of claim 18, wherein the formulation comprises about 1% to about 3% hydroxyethyl cellulose (HEC) on a weight (mg)/volume (ml) basis, or on a weight/weight (mg) basis.

20. The formulation of claim 18, wherein the formulation comprises about 2% hydroxyethyl cellulose (HEC) on a weight (mg)/volume (ml) basis, or on a weight/weight (mg) basis.

21. The formulation of claim 18, wherein the paraben is selected from the group consisting of methyl paraben, butyl paraben, ethyl paraben, heptyl paraben, and propyl paraben.

22. The formulation of claim 18, wherein the formulation comprises about 0.11% methyl paraben on a weight (mg)/volume (ml) basis, or on a weight/weight (mg) basis.

23. The formulation of claim 18 wherein the formulation comprises about 0.022% propyl paraben on a weight (mg)/volume (ml) basis, or on a weight/weight (mg) basis.

24. The formulation of claim 18, wherein the formulation is present in a syringe or catheter.

25. The formulation of claim 18, wherein the formulation is provided on a wound dressing.
